(19) United States
(12) Patent Application Publication
(10) Pub. No.: US 2007/0110612 A1
(43) Pub. Date: May 17, 2007

(54) External circulation apparatus

(75) Inventor: Akiyasu Ito, Kanagawa (JP)

Correspondence Address: BUCHANAN, INGERSOLL & ROONEY PC, POST OFFICE BOX 1404, ALEXANDRIA, VA, 22313-1404, US

(73) Assignee: Terumo Kabushiki Kaisha, Tokyo (JP)

(21) Appl. No.: 11/594,924

(22) Filed: Nov. 9, 2006

(30) Foreign Application Priority Data
Nov. 11, 2005 (JP) 2005-327320

(51) Int. Cl. A61M 37/00; A61M 1/00

(52) U.S. Cl. 422/44; 604/6.09; 604/6.11

(57) ABSTRACT

An external circulation apparatus capable of reliably detecting the level of a liquid in a foam reserving chamber of a defoaming device includes a pump for transferring and circulating blood externally of a body, a defoaming device for defoaming the blood externally circulated, and a controller for controlling the actions or operation of the centrifugal pump. The defoaming device includes a body portion having an internal space for the blood to flow in, a foam reserving chamber formed on the upper side of the body portion for receiving foam floating from the body portion, and a detector for detecting the liquid level of the blood in the foam reserving chamber. The detector includes a pair of electrode portions having at least a portion exposed to the inside of the foam reserving chamber, and a power feed unit for feeding electricity between the electrode portions. The controller controls the action of the centrifugal pump on the basis of the information obtained from the detector.

### FIELD OF THE INVENTION

The present invention generally relates to an external circulation apparatus. More particularly, the invention pertains to an external circulation apparatus that includes a blood pump for transferring and circulating blood externally of a body, a defoaming device for defoaming the blood externally circulated, and control means for controlling the actions of the blood pump.

### BACKGROUND DISCUSSION

In cardiosurgery operations, for example, a blood pump is activated to perform artificial lung external blood circulation in which blood is extracted from the vein (e.g., large vein) of a patient, subjected to gas exchange in an artificial lung, and then returned to the artery of the patient.

A circuit (an external circulation circuit) for the artificial lung external blood circulation is equipped with a defoaming device for removing (or separating) foam in the extracted blood. This defoaming device includes a housing or container body, and a filter member disposed in the housing for partitioning the housing interior into a blood inflow space for the blood to flow in and a blood outflow space for the blood to flow out. In this known defoaming device such as described in Japanese Application Publication No. 64-8562, foam is collected in the housing by applying centrifugal force to the blood and then the foam is removed.

Moreover, the defoaming device described above is usually equipped with a foam sensor for detecting the foam residing in the blood inflow space. One foam sensor includes an ultrasonic transmission unit and an ultrasonic reception unit disposed opposite the ultrasonic transmission unit with a gap between the ultrasonic transmission unit and an ultrasonic reception unit.

The ultrasonic reception unit receives the ultrasonic waves transmitted from the ultrasonic transmission unit and, making use of the fact that the liquid (blood) and the gas (foam) have different transmissivities to ultrasonic waves, the foam sensor detects whether the substance in the gap between the ultrasonic transmission unit and the ultrasonic reception unit is blood or foam. As a result, when foam is collected in the blood inflow space so that the liquid surface comes down to the position of the foam sensor, this can be detected by the foam sensor so that the gas (foam) can be prevented from being excessively accumulated in the blood inflow space.

If foam excessively accumulates in the blood inflow space, the foam may pass through the filter member. The foam may not be sufficiently or reliably removed, but may be released together with the blood that has passed through the filter member and may pass out of the defoaming device.

The foam sensor described above is a sensor which uses ultrasonic waves. This foam sensor using ultrasonic waves is liable to receive potential adverse influences of the environment, such as noises. Therefore, the foam sensor may erroneously detect that the liquid surface has dropped to the position of the foam sensor when in fact the liquid surface has not dropped to such position.

In the external circulation circuit, air in the circuit is replaced by physiological saline before the blood is circulated, that is before the cardiosurgery operations. As a result, the air in the external circulation circuit can be prevented from being sent to the human body.

The cardiosurgery operations are started after the external circulation circuit has been filled up with the physiological saline.

In the external circulation circuit filled up with the physiological saline, when the cardiosurgery operations are started, an interface is established between the physiological saline and the blood in the defoaming device due to the difference in the specific gravity between the physiological saline and the blood. When this interface goes up or rises to the position of the foam sensor which uses ultrasonic waves, erroneous detections frequently occur such that the foam sensor senses that the liquid surface has dropped to the position of the foam sensor, though the liquid surface has not in fact dropped to such position.

In addition, in this external circulation circuit, each time the erroneous operation or erroneous detection of the foam sensor occurs, the blood pump is interrupted, or the clamp for blocking the external circulation circuit midway is activated to stop the circulation of the blood so that the availability is lowered. As a result, the blood circulation in the patient may become unstable.

### SUMMARY

An external circulation apparatus comprises a blood pump for circulating blood externally of a body, a defoaming device for defoaming the externally circulated blood, and control means for controlling the actions or operations of the blood pump. The defoaming device includes a device body having an internal space for the blood to flow in, a foam reserving chamber formed on the upper side of the device body for temporarily reserving the foam floated from the device body, and detecting means for detecting the liquid level of the blood in the foam reserving chamber or information on the liquid level. The detecting means includes a first electrode portion having at least its portion exposed to the inside of the foam reserving chamber, a second electrode portion having at least a portion exposed to the inside of the device body or the foam reserving chamber, and a power feed unit for feeding electricity between the first electrode portion and the second electrode portion. The control means controls the operation of the blood pump on the basis of the information obtained from the detecting means.

The control means maintains the operation of the blood pump when a decision unit decides a conductive state exists between the first electrode portion and the second electrode portion through a liquid, and stops the operation of the blood pump when the decision unit decides the non-conductive state does not exist between the first electrode portion and the second electrode portion. The blood pump can be a centrifugal pump.

According to another aspect, an external circulation apparatus comprises a line through which blood is transferred to outside a body, a clamp for shielding a portion of the line, a defoaming device for defoaming the blood, and control means for controlling the operation or action of the clamp. The defoaming device includes a body portion having an internal space for the blood to flow in, a foam reserving chamber formed on the upper side of the body portion for temporarily reserving the foam floating from the device body, and detecting means for detecting the liquid level of the blood in the foam reserving chamber or information on the liquid level. the detecting means includes a first electrode portion having at least a portion exposed to the inside of the foam reserving chamber, a second electrode portion having at least a portion exposed to the inside of the body portion or the foam reserving chamber, and a power feed unit for feeding electricity between the first electrode portion and the second electrode portion. The control means controls the action or operation of the clamp on the basis of the information obtained from the detecting means.

The control means preferably includes a decision unit for deciding whether or not the first electrode portion and the second electrode portion conduct electricity through a liquid. Preferably, the current applied by the power feed unit is an AC current. In addition, the control means includes a conversion unit for converting the AC current between the first electrode portion and the second electrode portion into an AC voltage, and a rectification unit for full-wave rectifying the converted AC voltage.

The defoaming device preferably includes a first communication portion disposed in the body portion for communicating the crest of the body portion with the foam reserving chamber thereby to pass foam floated from the apparatus body therethrough, and a second communication portion disposed in the body portion communicating the circumferential wall portion of the body portion with the foam reserving chamber. The foam floated from the body portion flows through the first communication portion into the foam reserving chamber whereas the blood in the foam reserving chamber returns to the body portion through the second communication portion.

The defoaming device preferably includes a negative pressure chamber disposed on the upper side of the foam reserving chamber and connectable to deaeration means so that it is held under a negative pressure, and a filter member disposed to separate the foam reserving chamber and the negative pressure chamber for passing the gas in the foam reserving chamber therethrough but not the blood. The first electrode portion and the second electrode portion are preferably positioned in the vicinity of the lower portion of the foam reserving chamber.

The first electrode portion and the second electrode portion are preferably individually made of stainless steel.

According to the invention, the current between the paired electrodes disposed in the defoaming device can be detected to relatively reliably detect the liquid level of the liquid in the foam reserving chamber of the defoaming device.

Since the liquid level of the liquid in the foam reserving chamber of the defoaming device can be detected, the operation of the blood pump can be controlled according to the detection result so that the external circulation apparatus is capable of excellent operational ability.

Since the liquid level of the liquid in the foam reserving chamber of the defoaming device can be detected, moreover, the action or operation of the clamp can be controlled according to the detection result so that the external circulation apparatus is capable of excellent operational ability.

According to another aspect, a method of controlling circulation of blood comprises circulating external of a body blood which has been removed from the body, defoaming the blood to separate foam from the blood, determining whether a level of blood in a chamber containing the foam which has been separated from the blood is at or above a predetermined level by electrical conduction, and controlling circulation of the blood external of the body based on whether the level of the blood is determined to be at or above the predetermined level.

### BRIEF DESCRIPTION OF THE DRAWING FIGURES

FIG. 1 is a schematic diagram illustration of one embodiment of an external circulation apparatus disclosed herein.

FIG. 2 is a cross-sectional side view of a defoaming device forming a part of the external circulation apparatus shown in FIG. 1.

FIG. 3 is a bottom or lower face view of the defoaming device as seen from the direction of arrow A in FIG. 2.

FIG. 4 is a cross-sectional view taken along the section line B-B in FIG. 2.

FIG. 5 is a cross-sectional view taken along the section line C-C in FIG. 3.

FIG. 6 is a cross-sectional view taken along the section line C-C in FIG. 3.

FIG. 7 is a block diagram illustrating portions of the external circulation apparatus shown in FIG. 1.

FIG. 8 is a flow chart showing a control program of a control device of the external circulation apparatus shown in FIG. 1.

FIG. 9 is a cross-sectional view of the vicinity of an electrode portion of a defoaming device according to another embodiment of the external circulation apparatus.

### DETAILED DESCRIPTION

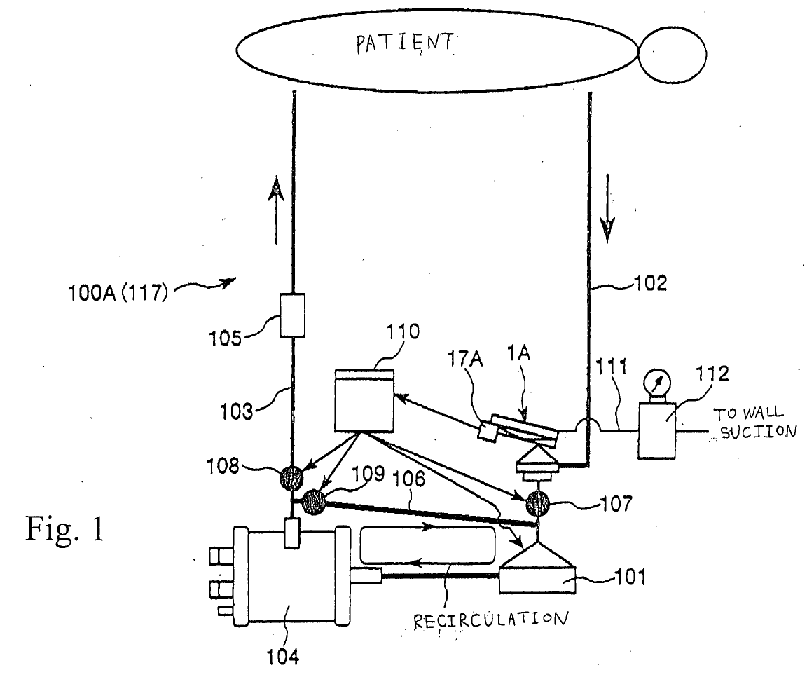

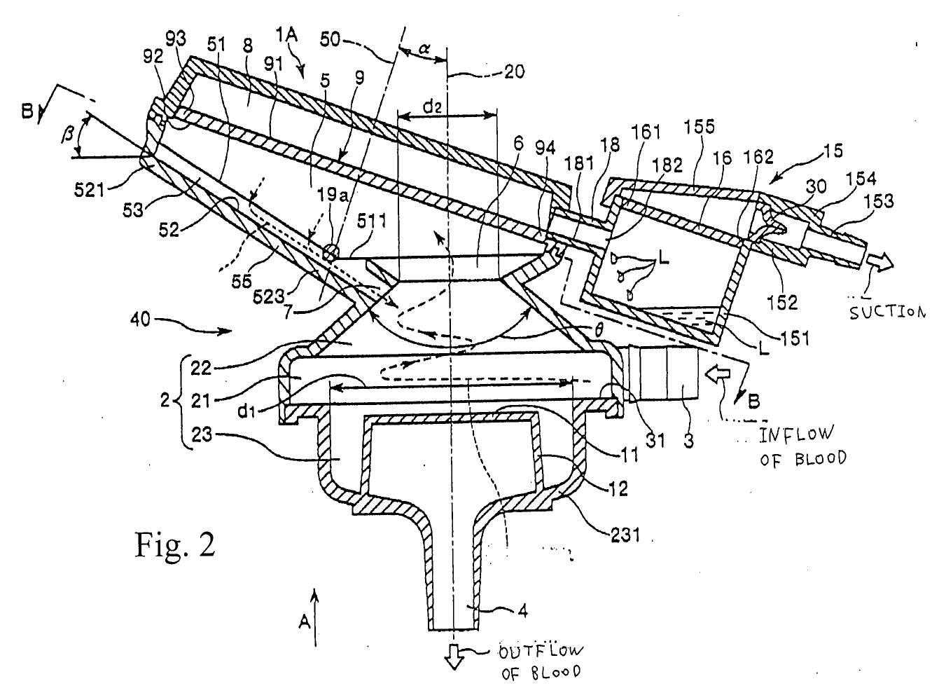

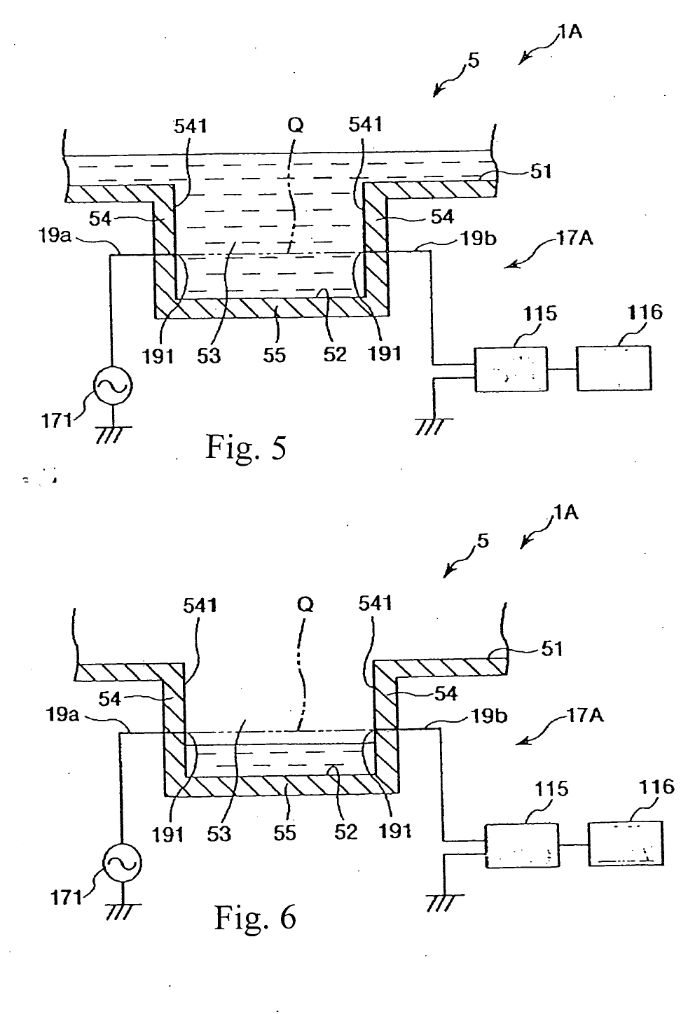

A schematic illustration of an embodiment of an external circulation apparatus disclosed herein is shown in FIG. 1, with additional aspects of the apparatus shown in FIGS. 2-8. For convenience of description, the upper sides in FIG. 2, FIG. 5 and FIG. 6 are referred to as “upper” or “upward” while the lower sides are referred to as “lower” or “downward”.

Referring to FIG. 1, the illustrated embodiment of the external circulation apparatus 100A disclosed herein includes a centrifugal pump (a blood pump) 101 for feeding or transferring blood, a blood extraction line 102 connecting the suction port of the centrifugal pump 101 and a patient, a blood feed line 103 connecting the discharge port of the centrifugal pump 101 and the patient, a defoaming device 1A disposed midway of the blood extraction line 102, an artificial lung 104 disposed along an intermediate portion of the blood feed line 103 for carrying out gas exchange with the blood (i.e., the addition of oxygen to the blood and the removal of carbon dioxide from the blood), a flow meter 105 disposed along an intermediate portion of the blood feed line 103, a recirculation line 106 for shortening the blood extraction line 102 near the suction port of the centrifugal pump 101 and the blood feed line 103 near the exit of the artificial lung 104, several clamps 107, 108, 109 for pinching/releasing tubes composing one or more of the lines to thereby close/open the passages, and a control device or control means 110 for controlling the operation of the clamps 107, 108, 109 and the centrifugal pump 101. Here, the circuit from the blood extraction line 102 to the blood feed line 103 of the external circulation apparatus 100A may be called the “external circulation circuit 117”.

The defoaming device 1A removes foam in the blood that is externally circulated. This defoaming device 1A can be employed for external circulation in which blood is not circulated to the heart of the patient and gas is not exchanged in the patient's body and in which blood circulation and gas exchange with the blood (i.e., oxygen addition and/or carbon dioxide removal) are carried out by the external circulation apparatus. This defoaming device 1A can also be employed for external circulation (or the auxiliary circulation) in which blood is circulated to the heart of the patient and gas is exchanged in the patient's body and in which blood circulation and gas exchange with the blood are carried out also by the external circulation apparatus.

As shown in FIG. 2, the defoaming device 1A includes a body or housing 40, a foam reserving chamber 5 disposed on the upper side of the body 40 (i.e., on the upper side of a swirling flow establishing chamber 2), a negative pressure chamber 8 disposed on the upper side of the foam reserving chamber 5, a liquid reserving chamber 15 communicating with the negative pressure chamber 8, for example through a connecting pipe 18, a first filter (a filter member or degasifying film) 9 disposed to isolate the foam reserving chamber 5 and the negative pressure chamber 8, a second filter 16 disposed in the liquid reserving chamber 15, and detecting means 17A (shown in FIG. 1) for detecting the liquid level Q of the blood in the foam reserving chamber 5.

The material(s) forming the body 40, the foam reserving chamber 5, the negative pressure chamber 8, the connecting pipe 18 and the liquid reserving chamber 15 is not particularly limited, but may, preferably, be a relatively hard resin material such as polycarbonate, acrylic resins, polyethylene terephthalate, polyethylene, polypropylene, polystyrene, polyvinyl chloride, acryl-styrene copolymer or acryl-butadiene-styrene copolymer. The material may also, preferably, be a substantially transparent material so that the state of internal blood or the like can be visibly confirmed.

The body 40 is equipped with the swirling flow establishing chamber 2 forming an internal space, an inlet port 3 for introducing blood into the swirling flow establishing chamber 2, an exit port 4 for discharging the blood in the swirling flow establishing chamber 2 to the outside of the defoaming device 1A, and a first communication portion 6 and a second communication portion 7 for affording communication between the swirling flow establishing chamber 2 and the foam reserving chamber 5.

The swirling flow establishing chamber 2 is a compartment having a rotor-shaped or annular internal space, i.e., an internal space having a generally circular cross-sectional shape, for establishing a swirling flow in the incoming blood. The defoaming device 1A is employed in a position (i.e., oriented) such that the center axis 20 of the swirling flow establishing chamber 2 is vertical (in the upward/downward direction). The plane normal to the center axis 20 of the swirling flow establishing chamber 2 is referred to as the “horizontal plane”.

This swirling flow establishing chamber 2 is formed to include a disc-shaped diametrically enlarged portion 21 positioned substantially at the same height as that of the inlet port 3, a frusto-conical portion 22 disposed on the upper side of (or above) the diametrically enlarged portion 21, and a trunk portion 23 disposed on the lower side of (or below) the diametrically enlarged portion 21.

The internal space of the frusto-conical portion 22 is generally frusto-conical in shape such that its internal diameter is gradually reduced upward. In the shown constitution, the internal space of the frusto-conical portion 22 is a frustum of a substantially complete circular cone. However, the internal space of the frusto-conical portion 22 need not be completely a frustum of a circular cone, but may have a rounded circumference in side view.

The internal space of the diametrically enlarged portion 21 is formed to possess a generally disc-shape configuration having a larger inner diameter than the internal diameter of the lower end of the frusto-conical portion 22.

The internal space of the trunk portion 23 is generally cylindrical in shape (or a generally columnar shape), having a smaller internal diameter than that of the diametrically enlarged portion 21. The lower portion of the trunk portion 23 is funnel shaped and is equipped at its lower end with the protruding exit port 4.

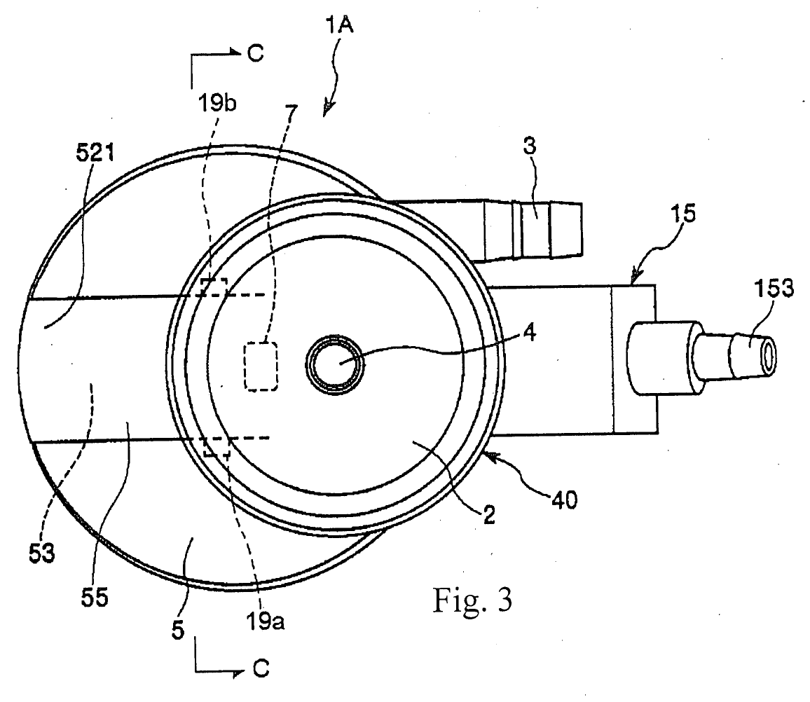

As depicted in FIG. 3, the inlet port 3 is disposed to protrude generally tangentially to the inner circumference of the diametrically enlarged portion 21 of the swirling flow establishing chamber 2.

With the disclosed embodiment of the body 40, blood that has flown from the inlet port 3 into the swirling flow establishing chamber 2 can reliably be formed into a swirling flow.

The foam reserving chamber 5 is a compartment for temporarily reserving the foam that have floated from the swirling flow establishing chamber 2. This foam reserving chamber 5 is filled up, when no foam is contained in the blood flowing into the swirling flow establishing chamber 2, with the blood.

The foam reserving chamber 5 has the generally disc-shaped internal space. The foam reserving chamber 5 has its upper portion covered by the first filter 9. Since the foam reserving chamber 5 is generally disc-shaped or possesses a generally circular shape, the area of the first filter 9 can be retained relatively large while reducing the charge or priming volume. In addition, foam residue at the time of charging the priming liquid can be relatively reliably prevented due to the absence of angled or sharp corners. Of course, while the described shape of the foam reserving chamber 5 provides certain functional or operational advantages, the foam reserving chamber 5 is not limited to the general disc shape, and may also be, for example, a polygonal plate shape.

This foam reserving chamber 5 has its center axis 50 offset (to the left side in FIG. 2) with respect to the center axis 20 of the swirling flow establishing chamber 2. As a result, the foam that has flown into the foam reserving chamber 5 is liable to gather on one side (or on the offset side, i.e., on the left side in FIG. 2) of the foam reserving chamber 5 so that the foam can efficiently pass through the first filter 9.

Moreover, the center axis 50 of the foam reserving chamber 5 is inclined with respect to the center axis 20 of the swirling flow establishing chamber 2. This inclination is so directed that portions of the foam reserving chamber 5 located farther from the center axis 20 of the swirling flow establishing chamber 2 are located at a higher height. Thus, relative to the illustration in FIG. 2, the foam reserving chamber 5 is inclined upwardly and to the left. As a result, foam that has flown into the foam reserving chamber 5 can be collected more smoothly and quickly on one side of the foam reserving chamber 5.

The angle α of inclination of the center axis 50 of the foam reserving chamber 5 with respect to the center axis 20 of the swirling flow establishing chamber 2 is not particularly limited, but is preferably about 0 to 50 degrees (preferably greater than zero degrees) and more preferably about 5 to 20 degrees.

The foam reserving chamber 5 has its bottom face 51 inclined so that the depth of the foam reserving chamber 5 increases towards the end closest to the swirling flow establishing chamber 2.

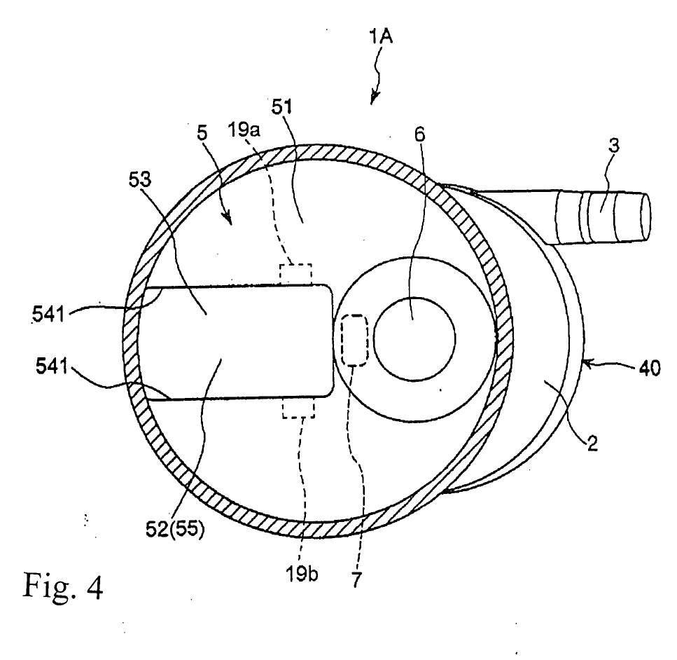

The frusto-conical portion 22 of the swirling flow establishing chamber 2 communicates near its crest (upper portion) with the foam reserving chamber 5 through the first communication portion 6. This first communication portion 6 is shaped as a circular opening formed in the bottom face 51 of the foam reserving chamber 5 as also shown in FIG. 4.

When the blood undergoes swirling flow in the swirling flow establishing chamber 2, the foam in the blood is collected, by the centrifugal force action, at the central portion due to the gas-liquid density difference. By virtue of buoyancy, the foam thus collected at the central portion floats and flows through the first communication portion 6 into the foam reserving chamber 5 as generally illustrated by dotted lines in FIG. 2.

The foam that flows into the foam reserving chamber 5 is collected, by buoyancy, toward the higher portion (i.e., the left side in FIG. 2) of the foam reserving chamber 5.

The swirling flow establishing chamber 2 and the foam reserving chamber 5 further communicate with each other through the second communication portion 7. This second communication portion 7 opens in the vicinity of the circumferential wall portion (inclined wall portion) at the left side of FIG. 2 of the frusto-conical portion 22. This second communication portion 7 provides communication between the foam reserving chamber 5 at the portion opposed to the first communication portion 6 through the central axis 50 and the circumferential wall portion of the frusto-conical portion 22.

Since the capacity of the foam reserving chamber 5 is naturally constant, the blood of the same capacity as that of the foam which has floated from the swirling flow establishing chamber 2 has to return, when it flows into the foam reserving chamber 5 through the first communication portion 6, in place of the foam from the foam reserving chamber 5 to the swirling flow establishing chamber 2.

By virtue of the second communication portion 7, the blood in the foam reserving chamber 5 can return through the second communication portion 7 into the swirling flow establishing chamber 2 (as indicated by the shorter dotted lines in FIG. 2), as the foam which has floated from the swirling flow establishing chamber 2 flows through the first communication portion 6 into the foam reserving chamber 5.

When the foam which has floated from the swirling flow establishing chamber 2 flows into the foam reserving chamber 5, a generally one-way flow of blood can be established along a route from the frusto-conical portion 22, to the first communication portion 6, to the foam reserving chamber 5, to the second communication portion 7 and to the frusto-conical portion 22, in that order, so that the foam in the swirling flow establishing chamber 2 can be introduced relatively efficiently, smoothly and quickly into the foam reserving chamber 5. Since the aforementioned one-way flow is established, it is possible to help prevent the possibility of blood residing in the foam reserving chamber 5. This thus contributes to achieving a secondary effect of making it difficult for blood coagulation to occur.

The second communication portion 7 communicates with the circumferential wall portion of the frusto-conical portion 22 so that the vicinity of the exit of the second communication portion 7 is closer to the center axis 20. Therefore, the swirling flow has a relatively slow speed near the exit of the second communication portion 7 so that the blood emanating from the second communication portion 7 can relatively smoothly enter the frusto-conical portion 22 while neither flowing backward nor disturbing the swirling flow.

The exit of the second communication portion 7 may be directed either normal in a top plan view to the circumferential wall of the frusto-conical portion 22, or tangential to the circumferential wall of the frusto-conical portion 22, i.e., in the direction of the swirling flow.

In the absence of the second communication portion 7, when the foam in the swirling flow establishing chamber 2 flows through the first communication portion 6 into the foam reserving chamber 5, blood returning from the foam reserving chamber 5 to the swirling flow establishing chamber 2 would pass through the first communication portion 6 in a direction opposite the foam. As a result, the flow in the vicinity of the first communication portion 6 may be disturbed and thus block the smooth passage of the foam.

In this embodiment, a groove 53 is formed in the bottom face 51 of the foam reserving chamber 5. This groove53 is on the side of the center axis 50 opposite the first communication portion 6. The bottom surface of the groove 53 forms an inclined face 52 of the groove 53 that continues to the second communication portion 7 such that it is inclined downward to the second communication portion 7 with respect to a horizontal plane. The inclined face 52 allows the blood in the foam reserving chamber 5 to flow down more smoothly and quickly into the second communication portion 7.

The angle β of inclination of the inclined surface 52 is not particularly restricted, but may preferably be 0 to 90 degrees (i.e., greater than zero degrees and less than or equal to 90 degrees), more preferably 5 to 40 degrees.

The first filter 9 is a film member, which permits the passage of air (or gas), but prevents the passage of blood. This first filter 9 (or the second filter 16) is preferably treated to have a hydrophobic surface or is a hydrophobic film.

Examples of materials for the hydrophobic film include polytetrafluoroethylene (PTFE), copolymer (FEP) of tetrafluoroethylene and hexafluoropropylene, copolymer (PFA) of tetrafluoroethylene and perfluoroalkylvinylether, polychlorotrifluoroethylene (PCTFE), polyvinylidene fluoride (PVDF), copolymer of (ETFE) ethylene and tetrafluoroethylene, copolymer (ECTFE) of ethylene and chlorotrifluoroethylene, or polypropylene (PP). The first filter 9 is preferably prepared by making those materials porous by the extension method, the micro-phase separation method, the electron beam etching method, the sintering method or the argon plasma particle method.

The hydrophobic treating method is not particularly limited. An example includes a method in which the surface of the first filter 9 is coated with a hydrophobic component material.

The first filter 9 is disposed vertically above the foam reserving chamber 5 with reference to the center axis 50 of the foam reserving chamber 5. The first filter 9 is inclined with respect to the plane (horizontal plane) that is normal to the center axis 20 of the swirling flow establishing chamber 2. The foam that has flown into the foam reserving chamber 5 is thus able to move along the inclined first filter 9 to one side (i.e., the left hand side in FIG. 2) of the foam reserving chamber 5 so that the foam can be collected more smoothly and quickly.

Moreover, the first filter 9 permits the passage of the gas in the foam reserving chamber 5, as described hereinbefore, so that any evaporation or water vapor from the foam reserving chamber 5 can pass through the first filter 9. Water vapor having passed through the first filter 9 condenses into a liquid L which can move along the inclined first filter 9 to the side opposed to the foams (i.e., to the right side in FIG. 2), that is to the side of the liquid reserving chamber 15. As a result, the liquid L can easily flow into the liquid reserving chamber 15.

The negative pressure chamber 8 is a compartment having an internal space which is separated from the foam reserving chamber 5 by the first filter 9. The internal space in the negative pressure chamber 8 possesses a planar or flat three-dimensional configuration. In the illustrated embodiment, this negative pressure chamber 8 is disposed concentrically with the foam reserving chamber 5. Thus, the center axis of the negative pressure chamber 8 is also inclined with respect to the center axis 20 of the swirling flow establishing chamber 2. As a result, the liquid L in the internal space of the negative pressure chamber 8 can move toward the liquid reserving chamber 15 so that it can relatively easily flow into the liquid reserving chamber 15.

The negative pressure chamber 8 does not admit the blood. In other words, the lower surface 92 of the first filter 9 contacts blood, but the upper surface 91 of the first filter 9 contacts blood.

The foam (or air) that is located in the foam reserving chamber 5 is sucked through the first filter 9 into the negative pressure chamber 8, by virtue of the negative pressure in the negative pressure chamber 8, and is discharged to the outside of the defoaming device 1A through a deaeration port 153 of the liquid reserving chamber 15.

As illustrated in FIG. 2, one end of the inclined negative pressure chamber 8 (i.e., the lower end at the right side of the negative pressure chamber 8) is connected to a connecting pipe 18 which protrudes from the negative pressure chamber 8.

In the illustrated embodiment, no step is established between the bottom face 181 of the connecting pipe 18 and the upper surface 91 of the first filter 9. In other words, it is preferable that the bottom surface 181 of the connecting pipe 18 forms a smooth continuation of the upper surface 91 of the first filter 9 so that the two are flush with one another, with the connecting pipe 18 and the liquid reserving chamber 15 being inclined at the same angle. The liquid L can thus be prevented from residing in the negative pressure chamber 8. That is, the liquid L can smoothly flow from the upper surface 91 of the first filter 9 to the bottom surface 181 of the connecting pipe 18 so that the liquid L can be reliably discharged to the liquid reserving chamber 15.

Moreover, the liquid reserving chamber 15 is connected or attached to the negative pressure chamber 8 through the connecting pipe 18.

The liquid reserving chamber 15 is equipped with a reservoir chamber body portion 151, a check valve mounting portion 152 for mounting a check valve 30, and the deaeration port 153 connected with a deaeration means. According to one example, the deaeration means can be the wall suction of an operation room. The wall suction is one of the medical piping facilities for gases such as oxygen, medical air or nitrogen or for suction, that is the pipes arranged in the wall of the operation room for suctioning (or discharging). The deaeration means may also be constituted by a vacuum pump(s).

In the illustrated embodiment, the reserve chamber body portion 151 is box-shaped. This reserving chamber body 151 is adapted to reserve or hold the liquid L which flows out of the negative pressure chamber 8 thereinto through the connecting pipe 18. As a result, the liquid L is reliably trapped or held in the reserving chamber body 151 so that the liquid L can be prevented from flowing out of the defoaming device 1A.

The check valve mounting portion 152 is a cylindrical portion disposed in the upper portion 155 of the reserving chamber body 151. Moreover, the check valve mounting portion 152 is inclined in the same direction as the protruding direction of the connecting pipe 18.

The deaeration port 153, which possesses a cylindrical shape, extends or protrudes from the end portion 154 of the check valve mounting portion 152. This arrangement of the deaeration port 153 helps facilitate the connection of the tube of the deaeration means to the deaeration port 153. The inside of the negative pressure chamber 8 is kept under a negative pressure so that gas (or air) in the negative pressure chamber 8 is discharged from the deaeration port 153.

The protruding direction (angle of inclination) of the deaeration port 153 is substantially identical to that of the connecting pipe 18 (or the check valve mounting portion 152). Moreover, the inner and outer diameters of the deaeration port 153 that are smaller than the inner and outer diameters respectively of the check valve mounting portion 152.

The second filter 16 and the check valve 30 are mounted in the liquid reserving chamber 15 thus constituted, there are mounted the second filter 16 and the check valve 30, the former of which. The second filter 16 is a film member made similar to that of the first filter 9 to permit the passage of air (or gas), but not the liquid L. The check valve 30 is a valve member which permits only the flow of gas to the deaeration means.

The second filter 16 is disposed between the negative pressure chamber 8 and the deaeration means. That is, the second filter 16 is disposed on the upper portion 155 side of the opening 182 in which the connecting pipe 18 of the reserving chamber body 151 opens to the reserving chamber body portion 151. As a result, the liquid L from the connecting pipe 18 can flow into the reserving chamber body portion 151 without any contact with the second filter 16. Therefore, the liquid L can be reliably held in the reserving chamber body 151 while being prevented from flowing to the outside of the defoaming device 1A.

In the illustrated embodiment, the second filter 16 is arranged generally in parallel with the first filter 9. That is, the second filter 16 is inclined at the same angle with respect to the horizontal direction as the first filter 9. Since the second filter 16 is mounted in such a position, any liquid L which touches the second filter 16 can relatively quickly leave the inclined second filter 16, which is disposed at an angle α of inclination). Thus, the second filter 16 can be prevented from being damaged in its air permeability (or its defoaming ability).

The second filter 16 is positioned on the upper side of the first filter 9 relative to its thickness direction, i.e., in the direction of the center axis 50. The second filter 16 has its uppermost end portion 161 positioned lower than the uppermost end portion 93 of the first filter 9, relative to a horizontal axis passing through the uppermost end portion 161. The lowermost end portion 162 of the second filter 16 is positioned substantially at the same height as the lowermost end portion 94 of the first filter 9 so that a horizontal axis passing through the lowermost end portion 162 of the second filter 16 also passes through the lowermost end portion 94 of the first filter 9.

Moreover, the first filter 9 and the second filter 16 are disposed at positions in which they are spaced apart from one another in the direction parallel to the center axis 50. That is, the first filter 9 and the second filter 16 lie in respective planes that are spaced apart from one another (i.e., the planes are not coplanar). As a result, the liquid L on the first filter 9 can be prevented from coming into contact with the second filter 16.

The check valve 30 is disposed between the deaeration port 153 and the second filter 16, i.e., in the check valve mounting portion 152. As a result, gas discharged or removed by the deaeration means can be reliably prevented from flowing backward into the negative pressure chamber 8 so that the gas can be removed from the defoaming device 1A. Moreover, the negative pressure state in the liquid reserving chamber 15 can be held at a relatively stable level.

In the illustrated embodiment, the check valve 30 is a duck bill valve as shown in FIG. 2. However, the check valve 30 is not limited in this regard as it may be formed as a different valve member allowing the flow of gas only to the side of the deaeration means.

In the illustrated defoaming device 1A, the frusto-conical portion 22 is disposed in the upper portion of the swirling flow establishing chamber 2, and foam can be collected through centrifugal force and the buoyancy so that the collected foam can be efficiently fed through the first communication portion 6 to the foam reserving chamber 5.

It has been found that foam, as collected at the center portion by the action of the swirling flow in the swirling flow establishing chamber 2, becomes a generally column-shaped lump, which is formed to have a diameter generally equal to the internal diameter d₂ of the first communication portion 6. If, therefore, the internal diameter d₂ of the first communication portion 6 is approximately equal to or larger than the maximum diameter of the swirling flow establishing chamber 2, the foam lump expands entirely into the swirling flow establishing chamber 2 thereby lowering the gas-liquid separating efficiency.

From this view point, it is preferable that the ratio of the internal diameter (or the maximum internal diameter) d₁ of the trunk portion 23 of the swirling flow establishing chamber 2 to the internal diameter d₂ of the first communication portion 6 is d₁:d₂=about 1:1 to 10:1, and is more preferably about 2:1 to 4:1.

The apex angle θ of the frusto-conical portion 22 is preferably 10 to 170 degrees, more preferably 30 to 150 degrees, and even more preferably 40 to 120 degrees.

If the apex angle θ of the frusto-conical portion 22 is excessively large, the frusto-conical portion 22 approaches a flattened shape having a small height and so it may be difficult to introduce the foam into the foam reserving chamber 5 by making effective use of the buoyancy. If the frusto-conical portion 22 has an excessively small apex angle θ, its height is increased to increase the charge.

A disc 11 is disposed in the trunk portion 23 of the swirling flow establishing chamber 2 and a connecting member 12 connects the disc 11 to the bottom portion of the swirling flow establishing chamber 2. The disc 11 acts to define the lower end of the foam lumps collected at the center portion. The disc 11 is disposed at a position normal to the center axis 20 of the swirling flow establishing chamber 2. The disc 11 is preferably disposed concentrically relative to the swirling flow establishing chamber 2, but may also be eccentrically disposed.

The disc 11 helps prevent the foam lumps from being formed below the disc 11 so that the collected foams can be more reliably prevented from flowing out of the exit port 4.

The upper face of the disc 11 is preferably positioned at the same height as or lower than the lower surface (end) 31 of the inlet port 3. As a result, the disc 11 does not block the formation of the swirling flow. The diameter of the disc 11 is preferably the same as or larger than the internal diameter of the first communication portion 6. As described above, the diameter of the foam lump is about as large as the internal diameter of the first communication portion 6. Therefore, the diameter of the disc 11 is made equal to or greater than the internal diameter of the first communication portion 6 so that the diameter of the disc 11 is made equal to or greater than the foam lump. Therefore, the foam lump can be more reliably prevented from being formed below the disc 11.

The disc 11 is fixed at the upper end portion of the connecting member 12. This connecting member 12 is a cylindrical member having an outer diameter substantially equal to that of the outer diameter of the disc 11, and its lower end is fixed on the bottom surface of the swirling flow establishing chamber 2. The circumferential wall of the connecting member 12 is provided with a plurality of slits or openings through which the blood flows from the outer circumferential side to the inner circumferential side of the connecting member 12 and further to the exit port 4.

Filters impermeable to the foam may be disposed in the slits or the openings of the connecting member 12. This connecting member 12 may also be formed as a plurality of spaced apart members or legs for supporting the disc 11.

The annular-shaped (or cylindrical) passage formed between the inner circumferential surface of the trunk portion 23 and the outer circumferential surfaces of the disc 11 and the connecting member 12 has a cross-sectional area larger than that of the passage of the inlet port 3. This arrangement can help reduce the flow resistance in that annular-shaped passage.

The defoaming device 1A is equipped with the detecting means 17A for detecting the liquid level Q of the blood in the foam reserving chamber 5. This detecting means 17A is equipped with a first electrode portion 19a, a second electrode portion 19b, and a power supply unit 171 for supplying electricity between the first electrode portion 19a and the second electrode portion 19b.

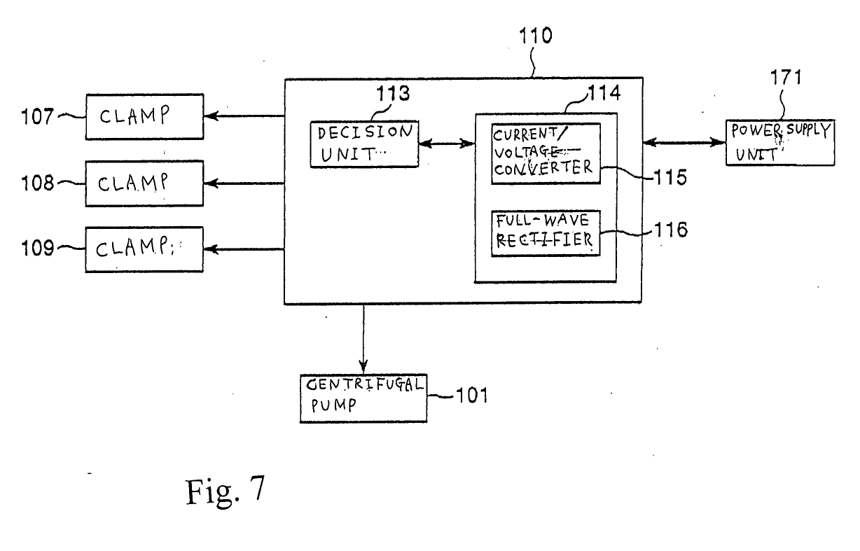

As shown in FIGS. 5 and 6, the first electrode portion 19a and the second electrode portion 19b are arranged in opposing or confronting relation to each other in the groove 53 of the foam reserving chamber 5. As shown in FIG. 2, moreover, the first electrode portion 19a and the second electrode portion 19b are positioned near the lower portion 523 of the inclined face 52 (or the lower portion of the foam reserving chamber 5). As described in more detail below and as schematically shown in FIG. 7, a processing unit 114 is also provided.

The first electrode portion 19a and the second electrode portion 19b are rod-shaped or plate-shaped and are made of a conductive material such as a metal material or a carbon material. The first electrode portion 19a and the second electrode portion 19b are also equipped with an insulating layer on their outer circumferences. The first electrode portion 19a and the second electrode portion 19b extend through the wall portion 54 of the groove 53 so that their end faces 191 are exposed to the wall face 541 (or into the groove 53).

When the liquid surface of the blood or liquid in the foam reserving chamber 5 is higher than the first electrode portion 19a and the second electrode portion 19b (or the liquid level Q), as shown in FIG. 5, the end faces 191 of the first electrode portion 19a and the second electrode portion 19b contact the blood. Since the blood generally has a conductivity, although low, the first electrode portion 19a and the second electrode portion 19b conduct electricity (referred to hereinafter as the “conductive state”) through the blood while a voltage is applied between the two electrodes.

When the liquid surface of the blood (or liquid) in the foam reserving chamber 5 is lower than the first electrode portion 19a and the second electrode portion 19b, or when the liquid surface is lower than the first electrode portion 19a or the second electrode portion 19b, as shown in FIG. 6, the end faces 191 of the first electrode portion 19a and the second electrode portion 19b do not contact the blood. At this time, the first electrode portion 19a and the second electrode portion 19b do not conduct electricity (referred to hereinafter as the “non-conductive state”).

In the non-conductive state, more specifically, the resistance between the first electrode portion 19a and the second electrode portion 19b becomes the maximum. When the electrodes come to the conductive state, on the other hand, the resistance between the first electrode portion 19a and the second electrode portion 19b becomes lower.

Thus, the first electrode portion 19a and the second electrode portion 19b can take the conductive and non-conductive states in accordance with the height of the liquid (blood) level. As a result, the external circulation apparatus 100A (or the detecting means 17A) can detect whether or not the liquid level is at or above the liquid level Q.

Examples of the materials for making the first electrode portion 19a and the second electrode portion 19b include stainless steel, titanium, a titanium alloy (e.g., a nickel-titanium alloy) or platinum, of which the stainless steel is preferred.

Because of excellent biological adaptability, stainless steel can be properly used for the first electrode portion 19a and the second electrode portion 19b to contact the blood.

Also, in case the first electrode portion 19a and the second electrode portion 19b are made of the stainless steel, their production cost can be reduced.

As shown in FIG. 5 and FIG. 6, the current applied between the first electrode portion 19a and the second electrode portion 19b is AC current. The AC current is less likely to hurt or cause damage to the cells in the blood than DC current.

In the embodiment described above, the current to be applied between the first electrode portion 19a and the second electrode portion 19b is desirably AC current. However, the applied current is not limited to AC current, as DC current may be applied.

In case the current applied between the first electrode portion 19a and the second electrode portion 19b is DC current, the resistance between the first electrode portion 19a and the second electrode portion 19b is measured. In the conductive state, the resistance is lower than that in the non-conductive state.

Therefore, the control device 110 is able to detect the liquid level Q by setting the threshold value at a predetermined resistance and deciding relative to the threshold value whether or not the measured resistance is large.

The clamp 107 is disposed in the blood extraction line 102 near the exit port 4 of the defoaming device 1A. The clamp 108 is disposed in the blood feed line 103 near the exit of the artificial lung 104. The clamp 109 is disposed in the recirculation line 106.

The clamps 107, 108 and 109 are individually controlled between their opened/closed states by the control device 110.

The clamps 107, 108 are normally controlled individually to be in the opened state. On the other hand, the clamp 109 is normally controlled to be in the closed state.

The deaeration port 153 of the defoaming device 1A is connected through a deaeration line 111 to the wall suction (or the deaeration means). A negative pressure regulator 112 is disposed at an intermediate point along the deaeration line 111. The negative pressure regulator 112 regulates the pressure in the negative pressure chamber 8.

As shown in FIG. 7, the control device 110 includes a decision unit 113 comprised of a CPU (Central Processing Unit), and the processing unit 114 for processing the AC current generated between the first electrode portion 19a and the second electrode portion 19b in the conductive state.

The processing unit 114 includes a current-voltage converter (or conversion unit) 115 and a full-wave rectifier (or rectification unit) 116.

The current-voltage converter 115 converts the AC current between the first electrode portion 19a and the second electrode portion 19b into an AC voltage. This current-voltage converter 115 is composed of, for example, two operation amplifiers, with one operation amplifier converting the AC current inputted from the first electrode portion 19a and the second electrode portion 19b into an AC voltage, and the other operation amplifier amplifying the converted AC voltage and outputting the amplified voltage to the full-wave rectifier 116.

The full-wave rectifier 116 rectifies, in the full-wave manner, the AC voltage converted by the current-voltage converter 115. This full-wave rectifier 116 includes a transformer having its input side connected with the current-voltage converter 115, and a diode connected with the output side of the transformer. When an AC voltage is applied to the input side (or the primary side) of the transformer, an AC voltage according to the winding ratio of that transformer is generated and is rectified by the diode so that it is outputted.

The full-wave rectifier 116 should not be limited to the aforementioned one using the transformer, but may be of a type which performs the full-wave rectification with a rectifying diode bridge and a capacitor or may be a full-wave rectifier utilizing an operation amplifier to correct the forward voltage drop of a diode. Alternatively, the analog signal of the current-voltage converter 115 may be subjected to an A/D conversion, and to a full-wave rectification by a digital signal processing.

Thus, in the conductive state, the external circulation apparatus 100A can establish the AC current and accordingly the AC voltage. In the non-conductive state, on the other hand, the AC current value is substantially zero, so that the according AC voltage is hard to generate.

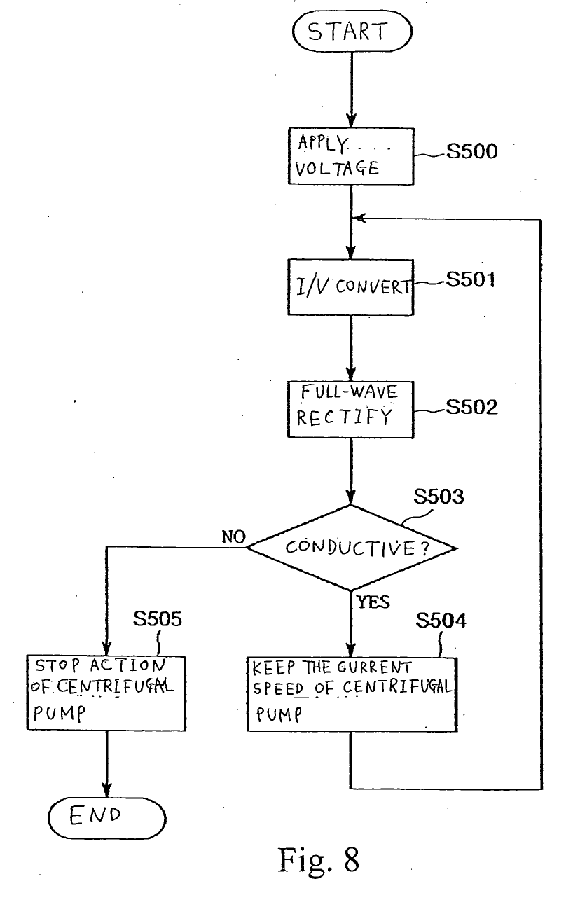

Referring to FIG. 8, the decision unit 113 decides on the basis of the output signal from the full-wave rectifier 116 whether or not the conductive state exists.

For example, the decision unit 113 compares the output signal from the full-wave rectifier 116 and the threshold value of the voltage (referred to as the “voltage threshold value”) stored in advance in the control device 110, and decides the conductive state exists if the output signal is at or above the voltage threshold value, and determines that the non-conductive state exists if the output signal is less than the voltage threshold value (or zero).

The control device 110 can thus reliably determine the conductive state and the non-conductive state between the first electrode portion 19a and the second electrode portion 19b. In accordance with this decision result, moreover, the control device 110 can relatively easily control the actions of the centrifugal pump 101.

The following is a description of the actions of the external circulation apparatus 100A.

Before the external circulation apparatus 100A is employed, the external circulation circuit 117 usually contains or is filled up with air. In the external circulation apparatus 100A, the air in the external circulation circuit 117 is replaced with physiological saline. This replacing method can be performed, for example, by activating the centrifugal pump 101. At this time, the clamps 107, 108, 109 are opened.

With the external circulation circuit 117 having its inside filled with physiological saline, the external circulation apparatus 100A is employed, for example, in cardiosurgery operations.

The control device 110 controls the clamps 107, 108 to normally be in the opened state and the clamp 109 to normally be in the closed state.

When the centrifugal pump 101 is activated to start the operations, the blood is extracted from the patient through the blood extracting catheter and flows through the blood extraction line 102 into the inlet portion 3 of the defoaming device 1A. In this defoaming device 1A, the foam in the blood is removed, as described hereinbefore. The blood from which the foam is removed is sent out from the exit port 4 of the defoaming device 1A through the centrifugal pump 101 into the artificial lung 104. In this artificial lung 104, the blood is subjected to a gas exchange operation in which oxygen is added and carbon dioxide is removed. The gas-exchanged blood is returned to the patient through the blood feed line 103 and the blood feed catheter.

In the defoaming device 1A having its inside filled up with the physiological saline, the centrifugal pump 101 is activated to extract the blood from the patient and to feed the blood back to the patient. As a result, an interface is established between the physiological saline and the blood in the foam reserving chamber 5. This interface rises as the physiological saline in the foam reserving chamber 5 is replaced by the blood. In the case of the detecting device mounted in the conventional defoaming device utilizing the transmissivity of ultrasonic waves, the detecting device erroneously detects the interface as the liquid level when the interface rises to reach the liquid level.

However, with the apparatus disclosed herein, by detecting the conductive state and the non-conductive state, the external circulation apparatus 100A is able to relatively reliably prevent the aforementioned erroneous detection from occurring.

In this external circulation apparatus 100A, when the amount of foam flowing together with the extracted blood into the defoaming device 1A is equal to the foam removing ability of the defoaming device 1A (or the defoaming means), the liquid level is stabilized (or balanced) at a position in the foam reserving chamber 5.

In this external circulation apparatus 100A, it is preferable that the liquid level of the blood in the foam reserving chamber 5 is positioned (or kept) in the state shown in FIG. 5, that is at or above the liquid level Q.

Therefore, the control device 110 stops the action of the centrifugal pump 101 when the liquid level falls from a position at or above the liquid level Q to a position below the liquid level Q, because the foam reserving chamber 5 is so filled up with foam as to make it difficult to remove the foam quickly and sufficiently from the defoaming device 1A. After this action of the centrifugal pump 101 is stopped, the defoaming device 1A is quickly cleared of the foam, and the centrifugal pump 101 is quickly activated again to restore the external circulation of the blood quickly.

While the centrifugal pump 101 is stopped, no new foam flows into the defoaming device 1A so that the foam in the defoaming device 1A is removed through the first filter 9 and the second filter 16 by the foam removing means (or deaeration means). As a result, the liquid level rises to a position higher than the liquid level Q.

The control flows (or programs) of the control device 110 of the external circulation apparatus 100A are described below primarily with reference to the flow chart of FIG. 8. When the external circulation is started, as described hereinbefore, the power supply unit 171 is activated (at Step S500).

Next, the AC current between the first electrode portion 19a and the second electrode portion 19b is converted into an AC voltage (at Step S501). Following this, the AC voltage converted at Step S501 is subjected to a full-wave rectification (at Step S502).

Next, the AC voltage full-wave rectified at Step S502 and the voltage threshold value stored in advance in the control device 110 are compared, as described hereinbefore, to decide (at Step S503) whether or not the conductive state is established between the first electrode portion 19a and the second electrode portion 19b.

If it is determined at Step S503 that the conductive state exists, the operation or active state (current speed) of the centrifugal pump 101 is maintained (at Step S504).

After the execution of Step S504, the flow chart returns to Step S501 and executes the subsequent steps sequentially.

If it is determined at Step S503 that the non-conductive state exists (or the conductive state does not exist), the operation of the centrifugal pump 101 is stopped (at Step S505).

By the control described above, the external circulation apparatus 100A can control the actions of the centrifugal pump 101 so that the foam may be reliably prevented from excessively residing in the foam reserving chamber 5 to thereby make the operational ability of the apparatus quite excellent.

As described above, if the non-conductive state is determined at Step S503, the action of the centrifugal pump 101 is stopped. However, the invention is not limited in this regard. For example, instead of stopping the operation of the centrifugal pump 101, the control device 110 may control the clamps 107, 108, 109 to close the clamps 107, 108 and open the clamp 109. As a result, the blood having left the artificial lung 104 returns again to the suction port of the centrifugal pump 101 through the recirculation line 106. As a result, the blood repeatedly circulates (or recirculates) through the annular passage including the centrifugal pump 101 and the artificial lung 104.

By these recirculations, it is possible to inhibit or prevent the foam in the defoaming device 1A from being sent to the patient and to suppress the damage of the blood in the centrifugal pump 101 even if the centrifugal pump 101 is continuously driven.

During this recirculation, the foam in the defoaming device 1A is relatively quickly removed, and then the ordinary external circulation state is restored by returning the clamps 107, 108 to the opened state and the clamp 109 to the closed state.

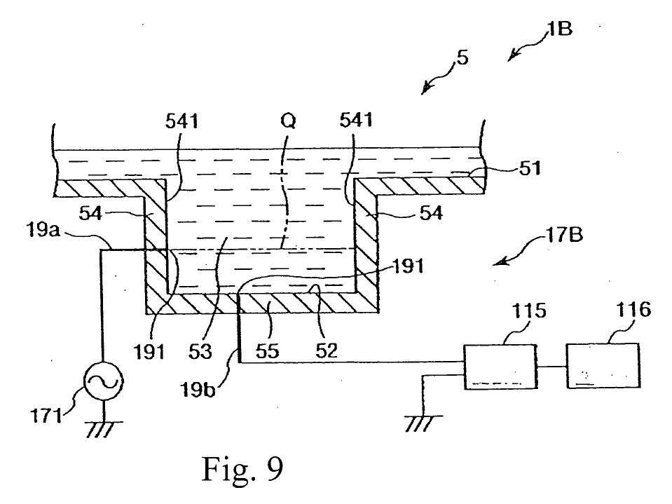

FIG. 9 is a cross-sectional diagram showing the vicinity of the electrode portion of the defoaming device of an external circulation apparatus according to a second embodiment. In FIG. 9, the illustrated circuit is equipped with the processing unit 114 described above.

With reference to this drawing, the second embodiment of the external circulation apparatus is described primarily with respect to the differences between this embodiment and the embodiment described above. A detailed description of features of the second embodiment that are the same as those associated with the first embodiment is not repeated.

This second embodiment is similar to the foregoing first embodiment, except that the place at which is mounted the second electrode portion is different.

As shown in FIG. 9, the second electrode portion 19b of the detecting means 17B of a defoaming device 1B extends so far through the bottom portion 55 of the groove 53 of the foam reserving chamber 5 that the end face 191 is exposed to the inclined surface 52.

Like the detecting means 17A of the first embodiment, the first electrode portion 19a extends through the wall portion 54 of the groove 53 so that the end face 191 is exposed at the wall surface 541.

As illustrated in FIG. 9, when the liquid surface of the blood (or the liquid) in the foam reserving chamber 5 is higher than the liquid level Q, the end faces 191 of the first electrode portion 19a and the second electrode portion 19b contact the blood. As a voltage is applied to the first electrode portion 19a and the second electrode portion 19b, electricity is conducted between the electrode portions by way of the blood.

When the liquid surface of the blood (or the liquid) in the foam reserving chamber 5 is lower than the liquid level Q, at least the end face 191 of the first electrode portion 19a does not contact the blood. At this time, the non-conductive state prevails between the first electrode portion 19a and the second electrode portion 19b.

Thus, the state between the first electrode portion 19a and the second electrode portion 19b can be conductive or non-conductive depending upon the height of the liquid level. In the external circulation apparatus 100A (or the detecting means 17B), therefore, it is possible to detect whether or not the liquid surface is at or above the liquid level Q.

Here, the second electrode portion 19b is mounted on the bottom portion 55 of the groove 53 of the foam reserving chamber 5 so that the end face 191 is exposed to the inside of the groove 53. However, other arrangements are also possible. For example, the electrode portion 19b may be mounted in the wall portion of the diametrically enlarged portion 21, in the wall portion of the frusto-conical portion 22 or in the wall portion of the trunk portion 23 so that the end face 191 is exposed to the internal space of the apparatus body 40.

In the known defoaming device described in the background portion, the sensor operates according to the principle that the blood and the gas have different transmissivities to ultrasonic waves in order to detect the liquid level in the foam reserving chamber. This sensor is equipped with an ultrasonic transmission unit for transmitting ultrasonic waves and an ultrasonic reception unit for receiving the ultrasonic waves sent from the ultrasonic transmission unit, with these units being arranged to confront each other. In this sensor, the ultrasonic transmission unit and the ultrasonic reception unit have to be rather specifically mounted at a portion of the foam reserving chamber so that the place for mounting the sensor is limited.

In the defoaming device 1B, however, one electrode portion (i.e., the second electrode portion 19b) need not always be mounted locally in the foam reserving chamber 5 with the other electrode portion (i.e., the first electrode portion 19a).

In the case of the liquid level sensor utilizing the ultrasonic waves, the transmission unit and the reception unit have to be arranged in line with one another. This thus requires an accurate positioning of the transmission unit and the reception unit. Also, in the case of this liquid level sensor, the faces of the transmission unit and the reception unit must be arranged parallel to one another and so the facing portions of defoaming device in which the transmission unit and the reception unit are arranged should be parallel. Thus, those portions of the defoaming device must be specifically and quite accurately constructed. It is also necessary for the individual confronting faces to be finished in a quite highly precise manner so that they are sufficiently smooth. Moreover, the ability to reduce the size of the liquid level sensor to a significant extent is somewhat limited.

This raises difficulties in the design (or manufacture) of the defoaming device so that the cost for manufacturing the die to produce the defoaming device is increased by designing it highly precisely, or the assembly cost (or manufacturing cost) of the defoaming device is increased.

On the other hand, in the defoaming device 1B disclosed here, the place for mounting the second electrode portion 19b is not so limited in that the second electrode portion 19b may be mounted anywhere in the defoaming device 1B so long as it contacts the liquid. This improves the degree of freedom for designing the defoaming device. In other words, it is not necessary to highly precisely produce parallelism between the two electrodes in the design of the defoaming device. Also, it is not necessary to precisely position the two electrodes, nor is it necessary to precisely set the roughness of the faces for mounting the two electrodes. It is also possible to utilize first and second electrode portions 19a, 19b that are relatively small in size.

As a result, it is possible to reduce the costs for manufacturing the mold of the defoaming device and for assembling (or manufacturing) the defoaming device.

In case a plate-shaped first electrode portion 19a is used and disposed vertically with respect to the liquid surface, the contact area of the first electrode portion 19a with the blood decreases as the liquid level of the blood is lowered. As a result, the current between the first electrode portion 19a and the second electrode portion 19b decreases. By detecting the current at this time in an analog manner, the position (or level) of the liquid surface at an arbitrary point of time can be detected.

Although the external circulation apparatus disclosed herein has been described by way of the illustrated embodiments, the invention is not limited in that regard. The individual portions constituting the external circulation apparatus can be replaced by other features capable of exhibiting the same or similar functions. Moreover, additional features or components may also be added.

The disclosed defoaming device is equipped with one detecting means. However, the invention is not limited in this regards as the defoaming device may be equipped with a plurality of detecting means.

In the case two detecting means are provided, for example, they are preferably arranged such that one detecting means detects a first liquid level whereas the other detecting means detects a second liquid level below the first one. In this case, the control device can make the following controls.

In case one detecting means detects that the liquid level of the blood has reached the first liquid level, the control device may control the action of the centrifugal pump so that the blood flowing into the defoaming device decreases. In case one detecting means detects that the liquid level of the blood reaches the first liquid level from the position between the first liquid level and the second liquid level, the control device may carry out a control to keep the operational state of the centrifugal pump at that time. On the other hand, in case the other detecting means detects that the liquid level of the blood reaches the second liquid level, the control device may also carry out a control to stop the operation of the centrifugal pump.

In case a plurality of detecting means are provided, the electricity may be selectively fed between pairs of electrode portions.

In the case of providing plural detecting means, moreover, each detecting means may be equipped with the power feed unit, or the plural detecting means may share one power feed unit.

Moreover, the external circulation apparatus (or the control device) may function to prevent an overcurrent between the electrodes. The method for preventing this overcurrent is not particularly limited, but the method may involve comparing the output signal from the full-wave rectifier and the threshold value of the voltage stored in advance in the control device to decide whether or not the output signal is higher than the threshold value of the voltage.

Moreover, the external circulation apparatus may have a self-diagnosing function (or the diagnostic function) to detect, before it is employed, whether or not the detecting means (or the power feed unit) is normally operating.

The liquid reserving chamber may be equipped with a discharge port for discharging the reserved liquid. As a result, the reserved liquid can be discharged from the liquid reserving chamber before it contacts with (or arrives at) the second filter.

This discharge portion may be ordinarily closed, but may be opened after, or example, an operation to eliminate the reserved liquid.

The liquid reserving chamber may be equipped with cooling means for cooling the inside of the liquid reserving chamber. As a result, steam can be reliably condensed in the liquid reserving chamber so that the steam can be reliably prevented from passing through the second filter. Here, the cooling means can be exemplified by disposing a heat sink around the body of the liquid reserving chamber or by mounting a Peltier element.

The principles, preferred embodiments and modes of operation have been described in the foregoing specification. However, the invention which is intended to be protected is not to be construed as limited to the particular embodiments disclosed. Further, the embodiments described herein are to be regarded as illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the claims, be embraced thereby.

What is claimed is:

1. An external circulation apparatus comprising:
a pump for circulating blood externally of a body;
a defoaming device for defoaming the externally circulated blood, the defoaming device comprising:
a body portion possessing an internal space adapted to receive the externally circulated blood;
a foam reserving chamber positioned above the body portion, the foam reserving chamber possessing an internal space for receiving foam floated from the body portion; and
detecting means for detecting a level of the blood in the foam reserving chamber, the detecting means comprising:
a first electrode portion having at least a portion exposed to the internal space of the foam reserving chamber;
a second electrode portion having at least a portion exposed to one of the internal space of the body portion and the internal space of the foam reserving chamber; and
a power feed unit for feeding electricity between said first electrode portion and said second electrode portion; and
control means for controlling operation of the blood pump based on the level of the blood in the foam reserving chamber detected by the detecting means.

2. An external circulation apparatus according to claim 1, wherein the control means comprises decision means for determining whether a conductive state exists between the first electrode portion and the second electrode portion through the blood, the control means maintaining operation of the blood pump when the decision means determines that the conductive state exists between the first electrode portion and the second electrode portion through the blood.

3. An external circulation apparatus according to claim 1, wherein the control means comprises decision means for determining whether a conductive state exists between the first electrode portion and the second electrode portion through the blood, the control means stopping operation of the blood pump when the decision means determines that the conductive state does not exist between the first electrode portion and said second electrode portion.

4. An external circulation apparatus according to claim 1, wherein the blood pump is a centrifugal pump.

5. An external circulation apparatus according to claim 1, wherein the control means comprises a decision unit for determining whether or not electricity is conducted between the first electrode portion and the second electrode portion through the blood.

6. An external circulation apparatus according to claim 1, wherein the power feed unit applies AC current to the first and second electrode portions.

7. An external circulation apparatus according to claim 6, wherein the control means comprises a conversion unit for converting the AC current between the first electrode portion and the second electrode portion into an AC voltage, and a rectification unit for full-wave rectifying the converted AC voltage.

8. An external circulation apparatus according to claim 1, wherein the defoaming device comprises:
a first communication portion disposed in the body portion and communicating an upper portion of body portion with the foam reserving chamber to permit passage of the foam from the body portion to the foam reserving chamber;
a second communication portion disposed in the body portion separate from the first communication port and communicating a circumferential wall portion of the body portion with the foam reserving chamber; and
wherein the foam floats from the body portion through the first communication portion into the foam reserving chamber while the blood in the foam reserving chamber returns to the body portion through the second communication portion.

9. An external circulation apparatus according to claim 1, wherein the defoaming device comprises:
a negative pressure chamber disposed on the upper side of the foam reserving chamber and connectable to deaeration means so that the negative pressure chamber is held under a negative pressure; and
a filter member disposed between the foam reserving chamber and the negative pressure chamber which permits passage of gas in the foam reserving chamber while preventing passage of blood.

10. An external circulation apparatus according to claim 1, wherein the first electrode portion and the second electrode portion are positioned in a lower portion of the foam reserving chamber.

11. An external circulation apparatus comprising;
a line through which blood from a body is conducted outside the body;
a clamp for closing off passage of the blood through at least a portion of the line;
a defoaming device connected to the line for defoaming the blood transferred outside the body;
the defoaming device comprising:
a body portion possessing an internal space for receiving the blood;
a foam reserving chamber positioned above the body portion for receiving foam floated from the body portion; and
detecting means for detecting a level of the blood in the foam reserving chamber;
the detecting means comprising:
a first electrode portion having at least a portion exposed to the internal space of the foam reserving chamber;
a second electrode portion having at least a portion exposed to one of the interior space of the body portion and the interior space of the foam reserving chamber; and
a power feed unit for feeding electricity between the first electrode portion and the second electrode portion; and
control means for controlling operation of the clamp based on the level of the blood in the foam reserving chamber detected by the detecting means.

12. An external circulation apparatus according to claim 11, wherein the control means comprises a decision unit for determining whether or not electricity is conducted between the first electrode portion and the second electrode portion through the blood.

13. An external circulation apparatus according to claim 11, wherein the power feed unit applies AC current to the first and second electrode portions.

14. An external circulation apparatus according to claim 13, wherein the control means comprises a conversion unit for converting the AC current between the first electrode portion and the second electrode portion into an AC voltage, and a rectification unit for full-wave rectifying the converted AC voltage.

15. An external circulation apparatus according to claim 11, wherein the defoaming device comprises:
a first communication portion disposed in the body portion and communicating an upper portion of body portion with the foam reserving chamber to permit passage of the foam from the body portion to the foam reserving chamber;
a second communication portion disposed in the body portion separate from the first communication port and communicating a circumferential wall portion of the body portion with the foam reserving chamber; and
wherein the foam floats from the body portion through the first communication portion into the foam reserving chamber while the blood in the foam reserving chamber returns to the body portion through the second communication portion.

16. An external circulation apparatus according to claim 11, wherein the defoaming device comprises:
a negative pressure chamber disposed on the upper side of the foam reserving chamber and connectable to deaeration means so that the negative pressure chamber is held under a negative pressure; and
a filter member disposed between the foam reserving chamber and the negative pressure chamber which permits passage of gas in the foam reserving chamber while preventing passage of blood.

17. An external circulation apparatus according to claim 11, wherein the first electrode portion and the second electrode portion are positioned in a lower portion of the foam reserving chamber.

18. An external circulation apparatus according to claim 11, wherein the first electrode portion and the second electrode portion are each made of stainless steel.

19. A method of controlling circulation of blood comprising:
circulating blood which has been removed from a body external of the blood;
defoaming the blood to separate foam from the blood;
determining whether a level of blood in a chamber containing the foam which has been separated from the blood is at or above a predetermined level by electrical conduction; and
controlling circulation of the blood external of the body based on whether the level of the blood is determined to be at or above the predetermined level.

20. The method according to claim 19, wherein the determination of whether the blood is at or above the predetermined level comprises detecting that the level of blood is at or above the predetermined level by conducting electric current between a first electrode portion positioned at the predetermined level and in contact with the blood and a second electrode in contact with the blood, with the electric current being conducted through the blood.

21. The method according to claim 19, wherein the controlling of the circulation of the blood external of the body comprises stopping operation of a pump which circulates the blood external of the body when it is detected that the level of the blood is below the predetermined level.

22. The method according to claim 19, wherein the controlling of the circulation of the blood external of the body comprises clamping a portion of a line through which the blood is circulated external of the body when it is detected that the level of the blood is below the predetermined level.
